SEQUENCE LISTING (2 sequences)

SEQ ID NO: 1 - DNA, 15418 bp, human
gcggccgcgagctctaatacgactcactatagggcgtcgactcgatcaatggaagatgaggcattgccgaagaaaagattaatggatttgaacacacagcaacagaaactacatgaagtgaaacacaggaaaaaaaagataaagaaacgaaaagaaaagggcatcagtgagcttcagcagaagttccatcggccttacatatgtgtaagcagaggccctgtaggagcagaggcagggggaaaatactttaagaaataatgtctaaaagtttttcaaatatgaggaaaaacataaaaccacagatccaagaagctcaacaaaacaaagcacaagaaacaggaagaaattaaaagttatatcacagtcaaattgctgaaaaccagcaacaaagagaatatcttaagagtatcagaggaaaagagattaatgacaggccaagaaacaatgaaaacaatacagatttcttgtaggaaacacaagacaaaagacattttttaaaaccaaaaggaaaaaaaatgctacattaaaatgttttttacccactgaaagtatatttcaaaacatattttaggccaggcttggtggctcacacctgtaatcccagcactttgggaggccaaggtgggtggatcgcttaaggtcaggagttcgagaccagcctggccaatatagcgaaaccccatctgtactaaaaacacaaaaattagctgggtgtggtgacacatgcctgtaatcccaggtactcaggaggctaaggcaggagaattgcttgaactgggaggcagaggtggtgagccaagattgcaccagtgcactccagccttggtgacagagtgaaactccatctcaaaaacaaacaaacaaaatacatatacataaatatatatgcacatatatatacatatataaatatatatacacatatataaatctatatacatatatacatatatacacatatataaatctatatacatatatatacatatataatatatttacatatataaatatatacatatataaatatacatatataaatacatatataaatatacatatataaatatacatatataaatatacatatataaatatatacatatataaatatacatatataaatatatatacatatataaatatataaatatacaagtatatacaaatatatacatatataaatgtatatacgtatatacatatatatataaatatataaaaaaacttttggctgggcacctttccaaatctcatggcacatataagtctcatggtaacctcaaataaaaaaacatataacagatacaccaaaaataaaaaccaataaattaaatcatgccaccagaagaaattaccttcactaaaaggaacacaggaaggaaagaaagaaggaagagaagaccatgaaacaaccagaaaacaaacaacaaaacagcaggagtaattcctgacttatcaataataatgctgggtgtaaatggactaaactctccaatcaaaagacatagagtggctgaatggacgaaaaaaacaagactcaataatctgttgcctacaagaatatacttcacctataaagggacacatagactgaaaataaaaggaaggaaaaatattctatgcaaatggaaaccaaaaaaagaacagaactagctacacttatatcagacaaaatagatttcaagacaaaaagtacaaaaagagacaaagtaattatataataataaagcaaaaagatataacaattgtgaatttatatgcgcccaacactgggacacccagatatatacagcaaatattattagaactaaggagagagagagatccccatacaataatagctggagacttcaccccgcttttagcattggacagatcatccagacagaaaatcaaccaaaaaattggacttaatctataatatagaacaaatgtacctaattgatgtttacaagacatttcatccagtagttgcagaatatgcattttttcctcagcatatggatcattctcaaggatagaccatatattaggccacagaacaagccattaaaaattcaaaaaaattgagccaggcatgatggcttatgcttgtaattacagcactttggggagggtgaggtgggaggatgtcttgagtacaggagtttgagaccagcctgggcaaaatagtgagaccctgtctctacaaacttttttttttaattagccaggcatagtggtgtgtgcctgtagtcccagctacttaggaggctgaagtgggaggatcacttgagcccaagagttcaaggctacggtgagccatgattgcaacaccacacaccagccttggtgacagaatgagaccctgtctcaaaaaaaaaaaaaaaaattgaaataatataaagcatcttctctggccacagtggaacaaaaccagaaatcaacaacaagaggaattttgaaaactatacaaacacatgaaaattaaacaatatacttctgaataaccagtgagtcaatgaagaaattaaaaaggaaattgaaaaatttatttaagcaaatgataacggaaacataacctctcaaaacccacggtatacagcaaaagcagtgctaagaaggaagtttatagctataagcagctacatcaaaaaagtagaaaagccaggcgcagtggctcatgcctgtaatcccagcactttgggaggccaaggcgggcagatcgcctgaggtcaggagttcgagaccagcctgaccaacacagagaaaccttgtcgctactaaaaatacaaaattagctgggcatggtggcacatgcctgtaatcccagctactcgggaggctgaggcaggataaccgcttgaacccaggaggtggaggttgcggtgagccgggattgcgccattggactccagcctgggtaacaagagtgaaaccctgtctcaagaaaaaaaaaaaagtagaaaaacttaaaaatacaacctaatgatgcaccttaaagaactagaaaagcaagagcaaactaaacctaaaattggtaaaagaaaagaaataataaagatcagagcagaaataaatgaaactgaaagataacaatacaaaagatcaacaaaattaaaagttggttttttgaaaagataaacaaaattgacaaacctttgcccagactaagaaaaaaggaaagaagacctaaataaataaagtcagagatgaaaaaagagacattacaactgataccacagaaattcaaaggatcactagaggctactatgagcaactgtacactaataaattgaaaaacctagaaaaaatagataaattcctagatgcatacaacctaccaagattgaaccatgaagaaatccaaagcccaaacagaccaataacaataatgggattaaagccataataaaaagtctcctagcaaagagaagcccaggacccaatggcttccctgctggattttaccaatcatttaaagaagaatgaattccaatcctactcaaactattctgaaaaatagaggaaagaatacttccaaactcattctacatggccagtattaccctgattccaaaaccagacaaaaacacatcaaaaacaaacaaacaaaaaaacagaaagaaagaaaactacaggccaatatccctgatgaatactgatacaaaaatcctcaacaaaacactagcaaaccaaattaaacaacaccttcgaaagatcattcattgtgatcaagtgggatttattccagggatggaaggatggttcaacatatgcaaatcaatcaatgtgatacatcatcccaacaaaatgaagtacaaaaactatatgattatttcactttatgcagaaaaagcatttgataaaattctgcacccttcatgataaaaaccctcaaaaaaccaggtatacaagaaacatacaggccaggcacagtggctcacacctgcgatcccagcactctgggaggccaaggtgggatgattgcttgggcccaggagtttgagactagcctgggcaacaaaatgagacctggtctacaaaaaacttttttaaaaaattagccaggcatgatggcatatgcctgtagtcccagctagtctggaggctgaggtgggagaatcacttaagcctaggaggtcgaggctgcagtgagccatgaacatgtcactgtactccagcctagacaacagaacaagaccccactgaataagaagaaggagaaggagaagggagaaaggagggagaagggaggaggaggagaaggaggaggtggaggagaagtggaaggggaaggggaagggaaagaggaagaagaagaaacatatttcaacataataaaagccctatatgacagaccgaggtagtattatgaggaaaaactgaaagcctttcctctaagatctggaaaatgacaagggcccactttcaccactgtgattcaacatagtactagaagtcctagctagagcaatcagataagagaaagaaataaaaggcatccaaactggaaaggaagaagtcaaattatcctgtttgcagatgatatgatcttatatctggaaaagacttaagacaccactaaaaaactattagagctgaaatttggtacagcaggatacaaaatcaatgtacaaaaatcagtagtatttctatattccaacagcaaacaatctgaaaaagaaaccaaaaaagcagctacaaataaaattaaacagctaggaattaaccaaagaagtgaaagatctctacaatgaaaactataaaatattgataaaagaaattgaagagggcacaaaaaaagaaaagatattccatgttcatagattggaagaataaatactgttaaaatgtccatactacccaaagcaatttacaaattcaatgcaatccctattaaaatactaatgacgttcttcacagaaatagaagaaacaattctaagatttgtacagaaccacaaaagacccagaatagccaaagctatcctgaccaaaaagaacaaaactggaagcatcacattacctgacttcaaattatactacaaagctatagtaacccaaactacatggtactggcataaaaacagatgagacatggaccagaggaacagaatagagaatccagaaacaaatccatgcatctacagtgaactcatttttgacaaaggtgccaagaacatactttggggaaaagataatctcttcaataaatggtgctggaggaactggatatccatatgcaaaataacaatactagaactctgtctctcaccatatacaaaagcaaatcaaaatggatgaaaggcttaaatctaaaacctcaaactttgcaactactaaaagaaaacaccggagaaactctccaggacattggagtgggcaaagacttcttgagtaattccctgcaggcacaggcaaccaaagcaaaaacagacaaatgggatcatatcaagttaaaaagcttctgcccagcaaaggaaacaatcaacaaagagaagagacaacccacagaatgggagaatatatttgcaaactattcatctaacaaggaattaataaccagtatatataaggagctcaaactactctataagaaaaacacctaataagctgattttcaaaaataagcaaaagatctgggtagacatttctcaaaataagtcatacaaatggcaaacaggcatctgaaaatgtgctcaacaccactgatcatcagagaaatgcaaatcaaaactactatgagagatcatctcaccccagttaaaatggcttttattcaaaagacaggcaataacaaatgccagtgaggatgtggataaaaggaaacccttggacactgttggtgggaatggaaattgctaccactatggagaacagtttgaaagttcctcaaaaaactaaaaataaagctaccatacagcaatcccattgctaggtatatactccaaaaaagggaatcagtgtatcaacaagctatctccactcccacatttactgcagcactgttcatagcagccaaggtttggaagcaacctcagtgtccatcaacagacgaatggaaaaagaaaatgtggtgcacatacacaatggagtactacgcagccataaaaaagaatgagatcctgtcagttgcaacagcatggggggcactggtcagtatgttaagtgaaataagccaggcacagaaagacaaacttttcatgttctcccttacttgtgggagcaaaaattaaaacaattgacatagaaatagaggagaatggtggttctagaggggtgggggacagggtgactagagtcaacaataatttattgtatgttttaaaataactaaaagagtataattgggttgtttgtaacacaaagaaaggataaatgcttgaaggtgacagataccccatttaccctgatgtgattattacacattgtatgcctgtatcaaaatatctcatgtatgctatagatataaaccctactatattaaaaattaaaattttaatggccaggcacggtggctcatgtccataatcccagcactttgggaggccgaggcggtggatcacctgaggtcaggagtttgaaaccagtctggccaccatgatgaaaccctgtctctactaaagatacaaaaattagccaggcgtggtggcacatacctgtagtcccaactactcaggaggctgagacaggagaattgcttgaacctgggaggcggaggttgcagtgagccgagatcatgccactgcactgcagcctgggtgacagagcaagactccatctcaaaacaaaaacaaaaaaaagaagattaaaattgtaatttttatgtaccgtataaatatatactctactatattagaagttaaaaattaaaacaattataaaaggtaattaaccacttaatctaaaataagaacaatgtatgtggggtttctagcttctgaagaagtaaaagttatggccacgatggcagaaatgtgaggagggaacagtggaagttactgttgttagacgctcatactctctgtaagtgacttaattttaaccaaagacaggctgggagaagttaaagaggcattctataagccctaaaacaactgctaataatggtgaaaggtaatctctattaattaccaataattacagatatctctaaaatcgagctgcagaattggcacgtctgatcacaccgtcctctcattcacggtgctttttttcttgtgtgcttggagattttcgattgtgtgttcgtgtttggttaaacttaatctgtatgaatcctgaaacgaaaaatggtggtgatttcctccagaagaattagagtacctggcaggaagcaggtggctctgtggacctgagccacttcaatcttcaagggtctctggccaagacccaggtgcaaggcagaggcctgatgacccgaggacaggaaagctcggatgggaaggggcgatgagaagcctgcctcgttggtgagcagcgcatgaagtgcccttatttacgctttgcaaagattgctctggataccatctggaaaaggcggccagcgggaatgcaaggagtcagaagcctcctgctcaaacccaggccagcagctatggcgcccacccgggcgtgtgccagagggagaggagtcaaggcacctcgaagtatggcttaaatctttttttcacctgaagcagtgaccaaggtgtattctgagggaagcttgagttaggtgccttctttaaaacagaaagtcatggaagcacccttctcaagggaaaaccagacgcccgctctgcggtcatttacctctttcctctctccctctcttgccctcgcggtttctgatcgggacagagtgacccccgtggagcttctccgagcccgtgctgaggaccctcttgcaaagggctccacagacccccgccctggagagaggagtctgagcctggcttaataacaaactgggatgtggctgggggcggacagcgacggcgggattcaaagacttaattccatgagtaaattcaacctttccacatccgaatggatttggattttatcttaatattttcttaaatttcatcaaataacattcaggagtgcagaaatccaaaggcgtaaaacaggaactgagctatgtttgccaaggtccaaggacttaataaccatgttcagagggatttttcgccctaagtactttttattggttttcataaggtggcttagggtgcaagggaaagtacacgaggagaggactgggcggcagggctatgagcacggcaaggccaccggggagagagtccccggcctgggaggctgacagcaggaccactgaccgtcctccctgggagctgccacattgggcaacgcgaaggcggccacgctgcgtgtgactcaggaccccataccggcttcctgggcccacccacactaacccaggaagtcacggagctctgaacccgtggaaacgaacatgacccttgcctgcctgcttccctgggtgggtcaagggtaatgaagtggtgtgcaggaaatggccatgtaaattacacgactctgctgatggggaccgttccttccatcattattcatcttcacccccaaggactgaatgattccagcaacttcttcgggtgtgacaagccatgacaacactcagtacaaacaccactcttttactaggcccacagagcacggcccacacccctgatatattaagagtccaggagagatgaggctgctttcagccaccaggctggggtgacaacagcggctgaacagtctgttcctctagactagtagaccctggcaggcactcccccagattctagggcctggttgctgcttcccgagggcgccatctgccctggagactcagcctggggtgccacactgaggccagccctgtctccacaccctccgcctccaggcctcagcttctccagcagcttcctaaaccctgggtgggccgtgttccagcgctactgtctcacctgtcccactgtgtcttgtctcagcgacgtagctcgcacggttcctcctcacatggggtgtctgtctccttccccaacactcacatgcgttgaagggaggagattctgcgcctcccagactggctcctctgagcctgaacctggctcgtggcccccgatgcaggttcctggcgtccggctgcacgctgacctccatttccaggcgctccccgtctcctgtcatctgccggggcctgccggtgtgttcttctgtttctgtgctcctttccacgtccagctgcgtgtgtctctgtccgctagggtctcggggtttttataggcataggacgggggcgtggtgggccagggcgctcttgggaaatgcaacatttgggtgtgaaagtaggagtgcctgtcctcacctaggtccacgggcacaggcctggggatggagcccccgccagggacccgcccttctctgcccagcacttttctgcccccctccctctggaacacagagtggcagtttccacaagcactaagcatcctcttcccaaaagacccagcattggcacccctggacatttgccccacagccctgggaattcacgtgactacgcacatcatgtacacactcccgtccacgaccgacccccgctgttttattttaatagctacaaagcagggaaatccctgctaaaatgtcctttaacaaactggttaaacaaacgggtccatccgcacggtggacagttcctcacagtgaagaggaacatgccgtttataaagcctgcaggcatctcaagggaattacgctgagtcaaaactgccacctccatgggatacgtacgcaacatgctcaaaaagaaagaatttcaccccatggcaggggagtggttggggggttaaggacggtgggggcagcagctgggggctactgcacgcaccttttactaaagccagtttcctggttctgatggtattggctcagttatgggagactaaccataggggagtggggatgggggaacccggaggctgtgccatctttgccatgcccgagtgtcctgggcaggataatgctctagagatgcccacgtcctgattcccccaaacctgtggacagaacccgcccggccccagggcctttgcaggtgtgatctccgtgaggaccctgaggtctgggatccttcgggactacctgcaggcccgaaaagtaatccaggggttctgggaagaggcgggcaggagggtcagaggggggcagcctcaggacgatggaggcagtcagtctgaggctgaaaagggagggagggcctcgagcccaggcctgcaagcgcctccagaagctggaaaaagcggggaagggaccctccacggagcctgcagcaggaaggcacggctggcccttagcccaccagggcccatcgtggacctccggcctccgtgccataggagggcactcgcgctgcccttctagcatgaagtgtgtggggatttgcagaagcaacaggaaacccatgcactgtgaatctaggattatttcaaaacaaaggtttacagaaacatccaaggacagggctgaagtgcctccgggcaagggcagggcaggcacgagtgattttatttagctattttattttatttacttactttctgagacagagttatgctcttgttgcccaggctggagtgcagcggcatgatcttggctcactgcaacctccgtctcctgggttcaagcaattctcgtgcctcagcctcccaagtagctgggatttcaggcgtgcaccaccacacccggctaattttgtatttttagtagagatgggctttcaccatgttggtcaggctgatctcaaaatcctgacctcaggtgatccgcccacctcagcctcccaaagtgctgggattacaggcatgagccactgcacctggcctatttaaccattttaaaacttccctgggctcaagtcacacccactggtaaggagttcatggagttcaatttcccctttactcaggagttaccctcctttgatattttctgtaattcttcgtagactggggatacaccgtctcttgacatattcacagtttctgtgaccacctgttatcccatgggacccactgcaggggcagctgggaggctgcaggcttcaggtcccagtggggttgccatctgccagtagaaacctgatgtagaatcagggcgcgagtgtggacactgtcctgaatctcaatgtctcagtgtgtgctgaaacatgtagaaattaaagtccatccctcctactctactgggattgagccccttccctatccccccccaggggcagaggagttcctctcactcctgtggaggaaggaatgatactttgttatttttcactgctggtactgaatccactgtttcatttgttggtttgtttgttttgttttgagaggcggtttcactcttgttgctcaggctggagggagtgcaatggcgcgatcttggcttactgcagcctctgcctcccaggttcaagtgattctcctgcttccgcctcccatttggctgggattacaggcacccgccaccatgcccagctaattttttgtatttttagtagagacgggggtgggggtggggttcaccatgttggccaggctggtctcgaacttctgacctcagatgatccacctgcctctgcctcctaaagtgctgggattacaggtgtgagccaccatgcccagctcagaatttactctgtttagaaacatctgggtctgaggtaggaagctcaccccactcaagtgttgtggtgttttaagccaatgatagaatttttttattgttgttagaacactcttgatgttttacactgtgatgactaagacatcatcagcttttcaaagacacactaactgcacccataatactggggtgtcttctgggtatcagcgatcttcattgaatgccgggaggcgtttcctcgccatgcacatggtgttaattactccagcataatcttctgcttccatttcttctcttccctcttttaaaattgtgttttctatgttggcttctctgcagagaaccagtgtaagctacaacttaacttttgttggaacaaattttccaaaccgcccctttgccctagtggcagagacaattcacaaacacagccctttaaaaaggcttagggatcactaaggggatttctagaagagcgacccgtaatcctaagtatttacaagacgaggctaacctccagcgagcgtgacagcccagggagggtgcgaggcctgttcaaatgctagctccataaataaagcaatttcctccggcagtttctgaaagtaggaaaggttacatttaaggttgcgtttgttagcatttcagtgtttgccgacctcagctacagcatccctgcaaggcctcgggagacccagaagtttctcgccccttagatccaaacttgagcaacccggagtctggattcctgggaagtcctcagctgtcctgcggttgtgccggggccccaggtctggaggggaccagtggccgtgtggcttctactgctgggctggaagtcgggcctcctagctctgcagtccgaggcttggagccaggtgcctggaccccgaggctgccctccaccctgtgcgggcgggatgtgaccagatgttggcctcatctgccagacagagtgccggggcccagggtcaaggccgttgtggctggtgtgaggcgcccggtgcgcggccagcaggagcgcctggctccatttcccaccctttctcgacgggaccgccccggtgggtgattaacagatttggggtggtttgctcatggtggggacccctcgccgcctgagaacctgcaaagagaaatgacgggcctgtgtcaaggagcccaagtcgcggggaagtgttgcagggaggcactccgggaggtcccgcgtgcccgtccagggagcaatgcgtcctcgggttcgtccccagccgcgtctacgcgcctccgtcctccccttcacgtccggcattcgtggtgcccggagcccgacgccccgcgtccggacctggaggcagccctgggtctccggatcaggccagcggccaaagggtcgccgcacgcacctgttcccagggcctccacatcatggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgcttcccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgggcctccccggggtcggcgtccggctggggttgagggcggccggggggaaccagcgacatgcggagagcagcgcaggcgactcagggcgcttcccccgcaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggtgaggaggtggtggccgtcgagggcccaggccccagagctgaatgcagtaggggctcagaaaagggggcaggcagagccctggtcctcctgtctccatcgtcacgtgggcacacgtggcttttcgctcaggacgtcgagtggacacggtgatcgagtcgactccctttagtgagggttaattgagctcgcggccgc

SEQ ID NO: 2 - DNA, 7498 bp, mouse
aagcttccagcaaaccagttagagctgagttgatgctctgaagaagagaaaatgtagagacggtactgaacaaataatgtctgggcaaacctcagacatgaaaatggaagacgtggaaatccagagaactctgagggaaaataaaacacaactccaggtcatcacgggactcatcaaactgctgaggtgcagccacagagaaaaatcttaaaatagcctagaacgatgcatgacacataaagcacagagaagacgaagctgagtctgtcttgtaggaacaacttgagaagacctaaaccactgcaatgagtgcattctgctaacttagaatttgctacccagttcagatccaaaaagggtttcacaaagttcaacacaaaacagtagcaggagtggctaagggggacacactgataggaattcagagaagtagggaatgctcatatggggacattacaaaatgtactttcatgttgcttaaatcattttaattgtcaaccacatcaagctaaataatgctttgaggttcataacatttggagattatgtctacactagcagagaaggcaccaataacatcccaattgctagattctcatagaatcatgagtcacaatggcagagacaggttctgagagtgtgtccttgttgtaaacagtatgctctacaaactaagttggctgcaatatcactaggcagtgttgtcccataagacaactatcacatatgtggtccagtgatgaccaaagcatcttttagcattttgcaaatgaagctcaaatcgaatatgactaagctcatgcagtacaaatcaaaggtacactgggatagtttaaaagatacatacttgtactggttagttttgtgtcagcttgacacagctggagttatcacagagaaaagagcttcagttgaggaaattcctccatgagatccagctatagggcattttctcaattagtgatcaaggggggaaggccccttgtgggtgggaccatctctgggctggtagtcttggttctataagagagcaggctgagcaagccaggagaagcaagccagtaaagaacatccctccatggcttctgcatcagctcctgctccctgacctgcttgagttccagttctaacttctttcagtgatgaacagcaatgtggaaatgaaagctgaataaaccctttcctccccattttgcttcttggtcatgatgtttgtgcaggaatagaaaccctgactaagacaatactataaaccctaaaagttgtaaaccaaacacatgtgtttccattaagccatcgtagaacaataagtactcaaccccaagtcacataactataatcccagcctttgaaaaccgggatcaggaattcaaggctagcctcatctatatgtaagattaaagcctgtttgggctgcatgagactttgtttcaaaaaaaaaaaaaaaaaagcaaacaggcaaaaacaaacacaagacaagacagatgtaaaatgaaggaggggtagatgggtcaagtagaaaatagcataggaaacgagtcaagtatagaagaggtggtagtaaccagatcatgcagaaggactcaaggccatctcctcacagtggcttaggtaggccttcctctgctcttgagcaggggcagagttgccgctttaaggaggggatcagtcacctttaagaactgaaaagctgaacagtcttctcaagtcagaagccagtggcttcatcttacacctctcttccttcccttgctactcatattggatctgatgatttgcccaacttggaagaaacatctcttctgaagggtttcacagacaccccatctttccgagaaaggaccgcataggctggccatccctgtgcttacaaaaggaataattaagaaacttaattccataagcaaatacaacctttccaagccccaagtggatgattttatcttactgtttttttatatctcatcaaataacttccaagggctcaaaaatccaaagatgtaaaaaaggaactgagctctgtttgccaagccatgaggattaaataatgacattcaaagagatttttgtgccctaagtactttttattggttttcatagatggtttaatgtgcaagatgaagcaaacagagatgggagtggtatcagcatggattaaggtggcagttgtgagggaggggtactgagagaacaggacaaggtaacctatctaaggagaggccaagttggcaagtgccagggacttctaagcccagaactagtacacattccttaggtgctgtttgggaagtcagggagtcaccagccttgggatctataaaagtgcatggtggcattcactcacatacttcctgagctgttcgatgttgatgaagtcgtgggtatgagactgttgtgtcagtgacaaactatgtaaatgagaatgattgtttccatcttgaccactaagacgtaaaccggttccagtgatctccaaacatggcaagctacagcagagcagcagccccatccagagccttgccctggttctgaatgggggagaatccagtgggagtcggttgctgccagcatgttggggtagaaggctggagcatgacaggtccccgaggatttcctgcttcctatatgggtagggatacttgaggtcctctcttctacctccttccctgcagggtttataacctctaccactgtctgtctctgggatagctcctagggtgcagcccctccccaaaaaggcctctccctggcctcatgtctctaagaacagctttctaaagcaggcctgttacacaaaggctcccttttcctggcttcatcgttgctggtagacaacttccactcgttttccacttcagtttcttctactctgttgttatttgattctgatgcttgaacccagggttgtgtagtcagcaagtgctaccccctccctcctcttctttgtttttttgaggcagggtctcattttgcccaagtggacctaaatttcagcatgtagctggcctggttttgaatgccttctcatcctgcctctacttcccaagagtagcttacaagtgtgcaccaccatgccccgcgatattcttatttttgagactgttttctatgctggtttctttggggaactacactaaggtagcttacaagtgtgcaccaccatgccccgcgatattcttatttttgagactgttttctatgctggtttctttggggaactacactaaggtagcttcattgttggcataaatttctcagttcaggcccatatctcctaagtagcagaactaagcaaatctcaaacaaacccctcaaaaagactgatgtccactaaacggacttctaaaatagctcctgtaatcctgagcatttacaaggcggcagacctcctataagggagtaaatatgaaaacgcgcctgttcaaatgctaggtcggtggatagaagcaatttcctcagaaagctgaaggcaccaaaggttatatttgttagcatttcagtgtttgccaaactcagctacagtagagatcacagattccctatttcccagagattcaaaattcagcagcccctctctaactatggctcagagtcgtgtcattacatatgccccaacaacaacccccacccctatcctacccccgcctcacacgtgcaagtactatcacagttgccaacctagcagagctgccatcctaaggtcgaggtcgccgctttggctgtgtgcacaggcaagcgccctcacccaatggccctggccttgctatgggtgcgtgagttgagatgatgctctggactctgaggtgaaggccactggaacagtgaaaaaagctaacgcagggcttttacctagtccccttcctttggtggtgggtgtttacggaacatatttgggatctgagtgtatggtcgcaccacaataaagccttaacctatatagtagaatttcagctgtaatcattaagaactgagattgccaccacccacctcactgtctgtgtcaaccacagcaggctggagcagtcagctcaggaacaggcaaaaccttaggtccctccgcctacctaaccttcaatacatcaaggataggcttctttgcttgcccaaacctcgccccagtctagaccacctggggattcccagctcagggcgaaaaggaagcccgagaagcattctgtagagggaaatcctgcatgagtgcgccccctttcgttactccaacacatccagcaaccactgaacttggccggggaacacacctggtcctcatgcaccagcattgtgaccatcaacggaaaagtactattgctgcgaccccgccccttccgctacaacgcttggtccgcctgaatcccgccccttcctccgttcccagcctcatctttttcgtcgtggactctcagtggcctgggtcctggctgttttctaagcacacccttgcatcttggttcccgcacgtgggaggcccatcccggccttgagcacaatgacccgcgctcctcgttgccccgcggtgcgctctctgctgcgcagccgataccgggaggtgtggccgctggcaacctttgtgcggcgcctggggcccgagggcaggcggcttgtgcaacccggggacccgaagatctaccgcactttggttgcccaatgcctagtgtgcatgcactggggctcacagcctccacctgccgacctttccttccaccaggtgggcctccaggcgggatccccatgggtcaggggcggaaagccgggaggacgtgggatagtgcgtctagctcatgtgtcaagaccctcttctccttaccaggtgtcatccctgaaagagctggtggccagggttgtgcagagactctgcgagcgcaacgagagaaacgtgctggcttttggctttgagctgcttaacgaggccagaggcgggcctcccatggccttcactagtagcgtgcgtagctacttgcccaacactgttattgagaccctgcgtgtcagtggtgcatggatgctactgttgagccgagtgggcgacgacctgctggtctacctgctggcacactgtgctctttatcttctggtgccccccagctgtgcctaccaggtgtgtgggtctcccctgtaccaaatttgtgccaccacggatatctggccctctgtgtccgctagttacaggcccacccgacccgtgggcaggaatttcactaaccttaggttcttacaacagatcaagagcagtagtcgccaggaagcaccgaaacccctggccttgccatctcgaggtacaaagaggcatctgagtctcaccagtacaagtgtgccttcagctaagaaggccagatgctatcctgtcccgagagtggaggagggaccccacaggcaggtgctaccaaccccatcaggcaaatcatgggtgccaagtcctgctcggtcccccgaggtgcctactgcagagaaagatttgtcttctaaaggaaaggtgtctgacctgagtctctctgggtcggtgtgctgtaaacacaagcccagctccacatctctgctgtcaccaccccgccaaaatgcctttcagctcaggccatttattgagaccagacatttcctttactccaggggagatggccaagagcgtctaaacccctcattcctactcagcaacctccagcctaacttgactggggccaggagactggtggagatcatctttctgggctcaaggcctaggacatcaggaccactctgcaggacacaccgtctatcgcgtcgatactggcagatgcggcccctgttccaacagctgctggtgaaccatgcagagtgccaatatgtcagactcctcaggtcacattgcaggtttcgaacagcaaaccaacaggtgacagatgccttgaacaccagcccaccgcacctcatggatttgctccgcctgcacagcagtccctggcaggtatatggttttcttcgggcctgtctctgcaaggtggtgtctgctagtctctggggtaccaggcacaatgagcgccgcttctttaagaacttaaagaagttcatctcgttggggaaatacggcaagctatcactgcaggaactgatgtggaagatgaaagtagaggattgccactggctccgcagcagcccgggtgagcatggctggtctccagctgaatgcattaggggcccagaaaagggagacaatgggtggcagtaacccaggtccccagtggtgtggtggctttatgcagtccgtggttggatgagttccatcttatggtctctgactccaagctccctccagctcgccttgcacaaactaagattcttgtccaagccctgggcaggttctcagggctggggacattgtggtgaacagataagcagacggggagcatggtggataggagttctggcacagtgcaccagagagagtctggaagcgctagtgagagctaatgtaagggcccgtggttcgccaaagaatgataaccccggactcaaatagtatgccaaagcaaggagcatttcattctgcagaaatcaagcatgcaggtggggggggggggttgctctcattccaagatggagagacaaccaagtatagattttaaggggatcgggggcctttatcttactccatctctaggggcattccattactggggcatggggttggaggttggaaactgttaatggggaggtctggaaacttgctgccccattgtccttgcttcaggctaggtagctgagtagcttctaatggcaggatagtttctgactagctgtctaaagtctggggtgtttgtttttttgttttttctagtaacttacttgcctgaacttgctcagtttttaggcctggtctcctggactgccaatttgaagcctattaaggagtcagcctgtctcactactccaggttatctataatccccctgtagaacggtacctcactgataacaatgacagaccaacataggaacccactatccttgtggtgcatgagtttcaaaggttcttctggtcctcccagtgtgcagatccatgcttaagctatggtcctcccagtgtgcagatccgtgcttaagctatggtcttgcagctgctcgatctacaaagggtagggtgaacgaaggaaagataaatgaaaaaaaaaaaactgtttcctacagtgaagatcgctgccccatcttagctatgagaagggactggggagtggagcctggtgcataaaagaggattgtgttacttggaaggctgcagagcctggactcctgtgccctccttgcctggttttctgggtttaatgttgaggttggccctctgtagtcactacctgaccccttccctttcagccaaccctccggttacaccctgtgcatgtatggaaggggccaaacgccctatcctgctctcccttccccaaaattcttaggatattaacaacttatggggaaaagatggtagagctatgtttacccaccatgtacttgggaagctccgaagtaagctt

(19) United States
(12) Patent Application Publication
(10) Pub. No.: US 2006/0199171 A1
(43) Pub. Date: Sep. 7, 2006

(54) Assays for TERT promoter modulatory agents

(76) Inventor: William H. Andrews, Reno, NV (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/349,734

(22) Filed: Feb. 7, 2006

Related U.S. Application Data
(60) Provisional application No. 60/651,037, filed on Feb. 7, 2005.

(51) Int. Cl. C12Q 1/00; C12Q 1/68

(52) U.S. Cl. 435/4; 435/6

(57) ABSTRACT

Methods and compositions for assaying an agent for TERT promoter modulatory activity are provided. In the subject methods, an agent is contacted with a normal cell under assay conditions that provide for a detectable phenotype, e.g., cell death, upon modulation of TERT promoter transcription control activity. Also provided are compositions, systems and kits thereof, as well as devices, that find use in practicing the subject methods. The subject invention finds use in assaying agents for TERT promoter modulatory activity, such as in a high throughput format.

### CROSS-REFERENCE TO RELATED APPLICATIONS

Pursuant to 35 U.S.C. § 119 (e), this application claims priority to the filing date of U.S. Provisional Patent Application Ser. No. 60/651,037 filed Feb. 7, 2005; the disclosure of which application is herein incorporated by reference.

### INTRODUCTION

### BACKGROUND OF THE INVENTION

Telomeres, which define the ends of chromosomes, consist of short, tandemly repeated DNA sequences loosely conserved in eukaryotes. For example, human telomeres consist of many kilobases of (TTAGGG)n together with various associated proteins. Small amounts of these terminal sequences or telomeric DNA are lost from the tips of the chromosomes during S phase because of incomplete DNA replication. Many human cells progressively lose terminal sequence with cell division, a loss that correlates with the apparent absence of telomerase in these cells. The resulting telomeric shortening has been demonstrated to limit cellular lifespan.

Telomerase is a ribonucleoprotein that synthesizes telomeric DNA. In general, telomerase is made up of two components: (1) an essential structural RNA (TR or TER) (where the human component is referred to in the art as hTR or hTER); and (2) a catalytic protein (telomerase reverse transcriptase or TERT) (where the human component is referred to in the art as hTERT). Telomerase works by recognizing the 3′ end of DNA, e.g., telomeres, and adding multiple telomeric repeats to its 3′ end with the catalytic protein component, e.g., hTERT, which has polymerase activity, and hTR which serves as the template for nucleotide incorporation. Both the catalytic protein component and the RNA template component are activity-limiting components.

Because of its role in cellular senescence and immortalization, there is much interest in the development of protocols and compositions for regulating telomerase activity. Of particular interest is the development of assays that detect agents that directly regulate the endogenous TERT, and specifically hTERT, promoter, e.g., in a high-throughput format.

### LITERATURE OF INTEREST

U.S. Pat. Nos. 5,972,605; 6,610,839 and 6,664,046 and published U.S. Application 2004/0072787; as well as WO 02/070668; WO 03/016474; WO 03/000916; WO 02/101010; WO 02/090571; WO 02/090570; WO 02/072787; WO 02/070668; WO 02/16658; WO 02/16657; and the references cited therein. Also of interest is Li et al., Rapid Inhibition of Cancer Cell Growth Induced by Lentiviral Delivery and Expression of Mutant—Template Telomerase RNA and Anti-Telomerase Short-interfering RNA,” Cancer Res. (Jul. 15, 2004) 64:4833-4840.

### SUMMARY OF THE INVENTION

Methods and compositions for assaying an agent for TERT promoter modulatory activity are provided. In the subject methods, an agent is contacted with a normal cell under assay conditions that provide for a detectable phenotype, e.g., cell death, upon modulation of TERT promoter transcription control activity. Also provided are compositions, systems and kits thereof, as well as devices, that find use in practicing the subject methods. The subject invention finds use in assaying agents for TERT promoter modulatory activity, such as in a high throughput format.

### DEFINITIONS

As used herein, the term “TERT promoter” includes any TERT genomic sequences capable of driving transcription in a telomerase activity positive cell. Thus, TERT promoters of the invention include without limitation cis-acting transcriptional control elements and regulatory sequences that are involved in regulating or modulating the timing and/or rate of transcription of a TERT gene. For example, the TERT promoter of the invention comprises cis-acting transcriptional control elements, including enhancers, promoters, transcription terminators, origins of replication, chromosomal integration sequences, 5′ and 3′ untranslated regions, exons and introns, which are involved in transcriptional regulation. These cis-acting sequences typically interact with proteins or other biomolecules to carry out (turn on/off, regulate, modulate, etc.) transcription.

As used herein, the terms “allele” or “allelic sequence” refer to an alternative form of a nucleic acid sequence (i.e., a nucleic acid corresponding to a TERT promoter, particularly, an hTERT promoter). Alleles result from mutations (i.e., changes in the nucleic acid sequence), and can produce differently regulated mRNAs. Common mutational changes that give rise to alleles are generally ascribed to natural deletions, additions, or substitutions of nucleotides. Each of these types of changes may occur alone, in combination with the others, or one or more times within a given gene, chromosome or other cellular nucleic acid. Thus, the term “TERT promoter” includes allelic forms of TERT promoter sequences, i.e., TERT cis-acting transcriptional control elements, including, e.g., the exemplary human and mouse sequences described herein. In alternative embodiments, the TERT promoter sequence comprises TERT sequences 5′ (upstream) of the translational start site (ATG). For example, in one embodiment, the hTERT promoter comprises residues 44 to 13545 of SEQ ID NO:01. Other embodiments include sequences starting within about one to 5 nucleotides of a translation start codon (for example in SEQ ID NO:01) and ending at about 50, 100, 150, 200, 250, 500, 1000, 2500 or 13500 nucleotides upstream of the translation start codon. Such embodiments can optionally include other regulatory sequences, such as, exon and/or intron sequences. hTERT promoters of the invention also include sequences substantially identical (as defined herein) to an exemplary hTERT promoter sequence of the invention, having the sequence set forth by SEQ ID NO:01. Similarly, mTERT promoters of the invention also include sequences substantially identical to an exemplary mTERT promoter sequence of the invention, having the sequence set forth by SEQ ID NO:02.

The term “heterologous” when used with reference to portions of a nucleic acid, indicates that the nucleic acid comprises two or more subsequences which are not found in the same relationship to each other in nature. For instance, the nucleic acid is typically recombinantly produced, having two or more sequences from unrelated genes arranged in a manner not found in nature; e.g., a promoter sequence of the invention operably linked to a polypeptide coding sequence that, when operably linked, does not reform the naturally occurring TERT gene. For example, the invention provides recombinant constructs (expression cassettes, vectors, viruses, and the like) comprising various combinations of promoters of the invention, or subsequences thereof, and heterologous coding sequences, many examples of which are described in detail below.

As used herein, “isolated,” when referring to a molecule or composition, such as, e.g., an hTERT promoter sequence, means that the molecule or composition is separated from at least one other compound, such as a protein, DNA, RNA, or other contaminants with which it is associated in vivo or in its naturally occurring state. Thus, a nucleic acid sequence is considered isolated when it has been isolated from any other component with which it is naturally associated. An isolated composition can, however, also be substantially pure. An isolated composition can be in a homogeneous state. It can be in a dry or an aqueous solution. Purity and homogeneity can be determined, e.g., using analytical chemistry techniques such as, e.g., polyacrylamide gel electrophoresis (PAGE), agarose gel electrophoresis or high pressure liquid chromatography (HPLC).

As used herein, the terms “nucleic acid” and “polynucleotide” are used interchangeably, and include oligonucleotides (i.e., short polynucleotides). They also refer to synthetic and/or non-naturally occurring nucleic acids (i.e., comprising nucleic acid analogues or modified backbone residues or linkages). The terms also refer to deoxyribonucleotide or ribonucleotide oligonucleotides in either single- or double-stranded form. The terms encompass nucleic acids containing known analogues of natural nucleotides. The term also encompasses nucleic acid-like structures with synthetic backbones. DNA backbone analogues provided by the invention include phosphodiester, phosphorothioate, phosphorodithioate, methyl-phosphonate, phosphoramidate, alkyl phosphotriester, sulfamate, 3′-thioacetal, methylene (methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NTYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (Strauss-Soukup (1997) Biochemistry 36:8692-8698), and benzyl-phosphonate linkages (Samstag (1996) Antisense Nucleic Acid Drug Dev 6:153-156).

As used herein, the term “operably linked” refers to a functional relationship between two or more nucleic acid (e.g., DNA) segments. Typically, it refers to the functional relationship of a transcriptional regulatory sequence to a transcribed sequence. For example, a promoter sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having coding or promoter sequences from different sources into an expression cassette or vector for expression of, e.g., a fusion protein; or, inducible, constitutive expression of a protein (i.e., a TERT promoter of the invention operably linked to a heterologous nucleotide, such as a polypeptide coding sequence).

As used herein, the “sequence” of a gene (unless specifically stated otherwise) or nucleic acid refers to the order of nucleotides in the polynucleotide, including either or both strands of a double-stranded DNA molecule, e.g., the sequence of both the coding strand and its complement, or of a single-stranded nucleic acid molecule. For example, in alternative embodiments, the TERT promoter of the invention comprises untranscribed, untranslated, and intronic TERT sequences, e.g., as set forth in the exemplary SEQ ID NO:01 and SEQ ID NO:02.

As used herein, the term “transcribable sequence” refers to any sequence which, when operably linked to a cis-acting transcriptional control element, e.g., a promoter, and when placed in the appropriate conditions, is capable of being transcribed to generate RNA, e.g., messenger RNA (mRNA).

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

The terms “identical” or percent “identity,” in the context of two or more nucleic acids or polypeptide sequences, refer to two or more sequences or subsequences that are the same or have a specified percentage of nucleotides (or amino acid residues) that are the same, when compared and aligned for maximum correspondence over a comparison window, as measured using one of the following sequence comparison algorithms or by manual alignment and visual inspection. This definition also refers to the complement of a sequence. For example, in alternative embodiments, nucleic acids within the scope of the invention include those with a nucleotide sequence identity that is at least about 60%, at least about 75-80%, about 90%, and about 95% of the exemplary TERT promoter sequence set forth in SEQ ID NO:01 (including residues 44 to 13544 of SEQ ID NO:01) or SEQ ID NO:02. Two sequences with these levels of identity are “substantially identical.” Thus, if a sequence has the requisite sequence identity to a TERT promoter sequence or subsequence of the invention, it also is a TERT promoter sequence within the scope of the invention. Preferably, the percent identity exists over a region of the sequence that is at least about 25 nucleotides in length, more preferably over a region that is at least about 50-100 nucleotides in length.

For sequence comparison, typically one sequence acts as a reference sequence, to which test sequences are compared. When using a sequence comparison algorithms test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identity for the test sequence(s) relative to the reference sequence, based on the designated or default program parameters. A “comparison window”, as used herein, includes reference to a segment of any one of the number of contiguous positions selected from the group consisting of from 25 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendrogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence (e.g., a TERT promoter sequence of the invention as set forth by. e.g., SEQ ID NO:01 or SEQ ID NO:02) is compared to another sequence to determine the percent sequence identity relationship (i.e., that the second sequence is substantially identical and within the scope of the invention) using the following parameters: default gap weight (3.00), default gap length weight (0.10), and weighted end gaps. PILEUP can be obtained from the GCG sequence analysis software package, e.g., version 7.0 (Devereaux (1984) Nuc. Acids Res. 12:387-395).

Another example of algorithm that is suitable for determining percent sequence identity (i.e., substantial similarity or identity) is the BLAST algorithm, which is described in Altschul (1990) J. Mol. Biol. 215:403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues, always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In one embodiment, to determine if a nucleic acid sequence is within the scope of the invention, the BLASTN program (for nucleotide sequences) is used incorporating as defaults a wordlength (W) of 11, an expectation (E) of 10, M=5, N=4, and a comparison of both strands. For amino acid sequences, the BLASTP program uses as default parameters a wordlength (W) of 3, an expectation (E) of 10, and the BLOSUM62 scoring matrix (see, e.g., Henikoff (1989) Proc. Natl. Acad. Sci. USA 89:10915).

The BLAST algorithm also performs a statistical analysis of the similarity between two sequences (see, e.g., Karlin (1993) Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### DESCRIPTION OF THE SPECIFIC EMBODIMENTS

Methods and compositions for assaying an agent for TERT promoter modulatory activity are provided. In the subject methods, an agent is contacted with a normal cell under assay conditions that provide for a detectable phenotype, e.g., cell death, upon modulation of TERT promoter transcription control activity. Also provided are compositions, systems and kits thereof, as well as devices, that find use in practicing the subject methods. The subject invention finds use in assaying agents for TERT promoter modulatory activity, such as in a high throughput format.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In further describing the invention, the subject methods are described first in greater detail, followed by a review of representative applications in which the subject methods find use, as well as a discussion of representative systems and kits that find use in practicing the subject methods.

Methods

As summarized above, the subject invention provides methods of determining whether an agent has TERT promoter modulatory activity. An agent is considered to have TERT promoter modulatory activity if its interaction with a TERT promoter causes a change in transcription activity, e.g., level (for example, in terms of transcribed copies of a coding sequence for a given period of time) of a nucleic acid sequence (e.g., a transcribable sequence, such as the coding sequence for TERT) operably linked to the promoter, e.g., as compared to a control (e.g., the transcription activity of an analogous TERT promoter/reporter nucleic acid construct not contacted with the agent of interest). The change that is observed is generally an increase in transcription of the operably linked nucleic acid, e.g., TERT coding sequence. In other words, the agent enhances transcription of the nucleic acid sequence operably linked to the TERT promoter. By enhance is meant that the expression level of the operably linked reporter nucleic acid sequence is increased by at least about 2 fold, usually by at least about 5 fold and sometimes by at least 25, 50, 100 fold and in particular about 300 fold or higher, as compared to a control, i.e., expression from an analogous or identical expression system that is not contacted with the agent in question. Alternatively, in cases where expression of the operably linked nucleic acid is so low that it is undetectable, expression of the operably linked nucleic acid is considered to be enhanced if expression is increased to a level that is easily detectable.

As reviewed above, in practicing the subject methods normal cells are contacted with a candidate agent under assay conditions that produce a detectable phenotype upon modulation of TERT promoter transcription control activity. The cell (also referred to herein as the target cell or test cell) with which the agent is contacted during practice of the subject methods is a normal cell that provides wild type conditions, e.g., a cell that normally lacks telomerase activity, e.g., an MRC5 cell, etc. In representative embodiments, the cell is a mammalian cell, where mammalian cells, of interest include, but are not limited to: murine, porcine, ovine, equine, rat, ungulates, dog, cat, monkey, and human cells, and the like. In many embodiments, the cell will be a human cell.

In practicing the subject methods, the target cell, as described above, is contacted with the candidate agent whose activity is to be tested. Contact of the candidate agent is achieved using any convenient protocol, such as introducing the agent into cell culture medium in which the target cell is present, etc. In many embodiments, normal cells are contacted with a candidate agent under assay conditions that produce a detectable phenotype upon enhancement of TERT promoter controlled expression mediated by the candidate agent, e.g., upon derepression of the TERT promoter by the candidate agent.

Any assay conditions that provide for a detectable phenotype in response to modulation of TERT promoter transcription control activity mediated by the candidate agent may be employed. The detectable phenotype may vary, where representative phenotypes include, but are not limited to: cell death; cell growth/proliferation; cell morphology; the production of new telomere sequences; changes in levels of telomere binding proteins; the presence or absence of binding proteins, e.g., transcriptions factors, that recognize new telomere sequences; changes in gene expression factors (e.g., resulting from presence of new telomere sequences and use of transcription factors as a result thereof, which modulates expression of other gene products); presence of restriction sites in new telomere sequences; changes in fluorescence polarization of the TR component (e.g., when bound to TERT); presence of a reporter gene; presence of a reporter sequence (e.g., directly or indirectly detectable) in a newly synthesized telomere sequence; detection of a TR/TERT complex, either directly or indirectly; and the like.

In certain representative embodiments, the detectable phenotype provided by the assay is cell death, and specifically the absence or occurrence thereof. For example, in certain embodiments the assay conditions are conditions that result in death of the cell when the candidate agent enhances, e.g., turns on, TERT expression. In these embodiments, the cell is contacted with the candidate agent under assay conditions that provide for cell death if the candidate agent modulates, and specifically enhances, expression of the TERT coding sequence as controlled by the target TERT promoter. In other representative embodiments where the detectable phenotype provided by the assay is cell death, the assay conditions are conditions that result in death of the cell when the candidate agent does not modulate, i.e., has no effect on, TERT expression. In these embodiments, the cell is contacted with the candidate agent under assay conditions that provide for cell death if the candidate agent does not modulate, and specifically does not enhance, expression of the TERT coding sequence as controlled by the target TERT promoter. As such, these assay conditions may be viewed as conditions that provide for cell death in the absence of TERT expression.

Representative assays that provide for cell death as an indication of the presence of TERT, and therefore activity of the TERT promoter, include antibody-based assays, in which antibodies that specifically bind to TERT mediate cell death of the TERT expressing cell. As such, in certain embodiments, an antibody-based assay may be employed to identify the occurrence of cell death. In these assays, the test cells may be mixed with an antibody that recognizes telomerase. Such an antibody is described in U.S. Pat. No. 6,639,057, the disclosure of which is herein incorporated by reference.

A first type of antibody-based assay of interest is a Complement Dependent Cytotoxicity (CDC) assay. In this representative assay, cells are mixed with a telomerase specific antibody and then mixed with serum containing complement that kills cells onto which the antibody has attached (i.e., telomerase expressing cells). Next, a reporter dye that distinguishes between live and dead cells, such as alamar blue (i.e., resazurin) which gets converted to resorufin by live cells and not by dead cells (Bioassays Systems, Cat # CQBL-05K), is contacted with the cells and the emission spectra of the cells is observed, e.g., one reads fluorescence emission by resorufin at 590 nm. Further details of such an assay are found in: Journal of Immunological Methods 202 (1997) 163-171.

A second representative antibody-based assay of interest is an Antibody Dependent Cellular Cytotoxicity (ADCC) assay. In such an assay, cells are mixed with antibody, e.g., as described above, and PBMC cells which kill cells in which the antibody has attached (i.e., telomerase expressing cells). Next, the cells are screened for cell death, e.g., with a lactate dehydrogenase activity assay (Roche Cat. Number 1644793). Such an assay is described in: Journal of Immunology 164 (2000) 4178-4184.

In yet other embodiments, a CTL assay may employed in which activated T-Cells (Cytotoxic Lymphocytes) that recognize telomerase expressing cells are produced as described in U.S. Pat. No. 6,440,735, the disclosure of which is herein incorporated by reference. These activated T-cells are then added to the cells being assayed. Cells expressing telomerase are killed. The cells can then be screened for the presence of cell death, e.g., as described above with alamar blue or the lactate dehydrogenase assay.

As reviewed above, other embodiments include contacting the test cell with the candidate agent under assay conditions that provide for cell death in the absence of TERT. In certain of these embodiments, an “Anti-Apoptosis” assay may be performed. Such an assay may be based on the finding by Rahman et al, Oncogene. 2004 Dec. 20; which reports that hTERT antagonizes p53-induced apoptosis independently of telomerase activity. In such an assay, an inducer of apoptosis, including but not limited to: Actinomycin D (cat #A9415), Anisomycin from Steptomyces griseolus (cat #A9789), Camptothecin (cat #C9911), or Vinblastine (cat #V1377) (all available from Sigma), is added to test cells that have been contacted with the agent. In such an assay, cells expressing hTERT would not go through apoptosis because the hTERT protein prevents it. The cells can then be screened for the present of cell death, e.g., as described above with alamar blue or the lactate dehydrogenase assay.

Other assays for dead or live cells that can be included in at least some of the assays described above include, but are not limited to: Caspase assays for the presence of apoptosis (such as Caspase 3 and 7 activity measurement: Caspase-Glo 3/7 Assay, cat.# G8092 (Promega); Caspase 8 activity measurement: Caspase-Glo 8 Assay, cat.# G8202 (Promega); Caspase 9 activity measurement: Caspase-Glo 9 Assay, cat.# G8212 (Promega); etc.); viability and proliferation assays, such as ATPlite, cell viability homogenous assay cat.# 6016947 (PerkinElmer), etc.; cell death detection assays, such as Cell Death Detection ELISAPLUS cat.# 1 920 685 (Roche Applied Science); Propidium Iodide assay (MTG, Inc. Product number M0795); etc.

Following contact of the candidate agent and the target cell, the phenotype of cell is evaluated or assessed to determine the promoter modulatory activity of the candidate agent. This step of assessing or evaluating the phenotype of the cell will necessarily vary depending on the nature of the phenotype that is induced by the presence of both TERT and the modified TR component in the cell. This step of the subject methods may include either a qualitative or quantitative evaluation of the phenotype, and may or may not include use of one or more reference or controls, as may be desired.

In certain embodiments, the subject methods are performed in a high throughput (HT) format. In the subject HT embodiments of the subject invention, a plurality of different compounds are simultaneously tested. By simultaneously tested is meant that each of the compounds in the plurality are tested at substantially the same time. Thus, at least some, if not all, of the compounds in the plurality are assayed for their effects in parallel. The number of compounds in the plurality that are simultaneously tested is typically at least about 10, where in certain embodiments the number may be at least about 100 or at least about 1000, where the number of compounds tested may be higher. In general, the number of compounds that are tested simultaneously in the subject HT methods ranges from about 10 to 10,000, usually from about 100 to 10,000 and in certain embodiments from about 1000 to 5000. A variety of high throughput screening assays for determining the activity of candidate agent are known in the art and are readily adapted to the present invention, including those described in e.g., Schultz (1998) Bioorg Med Chem Lett 8:2409-2414; Weller (1997) Mol Divers. 3:61-70; Fernandes (1998) Curr Opin Chem Biol 2:597-603; Sittampalam (1997) Curr Opin Chem Biol 1:384-91; as well as those described in published U.S. application 20040072787 and issued U.S. Pat. No. 6,127,133; the disclosures of which are herein incorporated by reference.

Testing of a candidate agent according to the invention as described above readily determines whether or not an agent has TERT promoter modulatory activity. As mentioned above, an agent is considered to have TERT promoter modulatory activity if its interaction with TERT promoter causes a change in transcription activity, e.g., level (for example, in terms of transcribed copies for a given period of time), of a nucleic acid sequence (i.e., transcribable sequence) operably linked to the promoter, e.g., as compared to a control (e.g., the transcription activity of an analogous TERT promoter/reporter nucleic acid construct not contacted with the agent of interest). The change that is observed may be an increase or decrease of TERT transcription. In other words, the agent may enhance or inhibit transcription of TERT. By enhance is meant that the expression level of TERT is increased by at least about 2 fold, usually by at least about 5 fold and sometimes by at least 25, 50, 100 fold and in particular about 300 fold or higher, as compared to a control, i.e., expression from an analogous or identical expression system that is not contacted with the agent in question. Alternatively, in cases where expression of TERT is so low that it is undetectable, expression of TERT is considered to be enhanced if expression is increased to a level that is easily detectable. By inhibit is meant that the expression level of the TERT is decreased by at least about 2 fold, usually by at least about 5 fold and sometimes by at least 25, 50, 100 fold and in particular about 300 fold or higher, as compared to a control, i.e., expression from an analogous or identical expression system that is not contacted with the agent in question.

Utility

A variety of different candidate agents may be screened by the above methods. Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The subject assays find use in any application where it is desired to determine whether a candidate agent has TERT promoter modulatory activity. Specifically, the subject assays find use in applications where one wishes to determine whether an agent has TERT promoter activator or enhancer activity. In representative embodiments, the methods provide for identification of agents which have human TERT promoter modulatory activity.

Agents identified in the above screening assays that inhibit repression of TERT transcription find use in the methods of enhancement of TERT expression, e.g., in the treatment of disease conditions, in research applications, etc., where representative specific applications include those described in United States Published Applications: 20030211965; 20030171326; 20030104420; 20030050264; and 20020193289; the disclosures of which are herein incorporated by reference.

Kits

Also provided are kits that find use in practicing the subject methods, as described above. For example, in some embodiments, kits for practicing the subject methods include at least a test cell as described above, or elements for constructing the same, e.g., expression vectors, etc. Furthermore, additional reagents that are required or desired in the protocol to be practiced with the kit components may be present, which additional reagents include, but are not limited to: aqueous mediums, culture mediums, and the like. The kits may also include reference or control elements, e.g., that provide calibration signals or values for use in assessing the observed signal generated by an assay performed with the kit components. The kit components may be present in separate containers, or one or more of the components may be present in the same container, where the containers may be storage containers and/or containers that are employed during the assay for which the kit is designed.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Systems

Also provided are systems that find use in practicing the subject methods, as described above. For example, in some embodiments, systems for practicing the subject methods include at least a test cell as described above. Furthermore, additional reagents that are required or desired in the protocol to be practiced with the system components may be present, which additional reagents include, but are not limited to: aqueous mediums, culture mediums, and the like. The systems may also include reference or control elements, e.g., that provide calibration signals or values for use in assessing the observed signal generated by an assay performed with the system components. The systems generally also include one or more candidate agents.

Devices

Also provided are high throughput (HT) devices that find use in practicing the subject methods, particularly HT embodiments thereof. The high throughput devices may have any convenient configuration, and generally include a plurality of two or more fluid containment elements in which assays can take place, agent administration elements and signal detection elements. For example, representative HT devices of the subject invention include a plate or substrate having a plurality of fluid-containing wells, reagent-adding equipment responsive to a computer for adding reagent, e.g., candidate agent, to the wells, measurement equipment for measuring at least one attribute of the sample or cells contained by the wells (e.g., for phenotype evaluation) and moving equipment which is responsive to the computer for aligning one of the wells first with the reagent-adding component, then with the measurement device, as further described in U.S. Pat. No. 6,127,133, the disclosure of which is herein incorporated by reference. Also of interest are the devices described in U.S. Pat. Nos. 6,468,736 and 5,989,835; as well as U.S. Provisional Application Ser. No. 60/618,484; the disclosures of which are herein incorporated by reference. A feature of the HT devices of the present invention is that they include in at least one fluid containment element containing a target cell as described above.

It is evident from the above results and discussion that the subject invention provides for greatly improved assays for determining the TERT promoter modulatory activity of a candidate agent. A particularly important advantage provided by certain embodiments of the subject invention is that the modulatory activity of the candidate agent is assessed on a native TERT gene present in a cell. Accordingly, the subject invention represents a significant contribution to the art.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of determining whether an agent modulates transcription control activity of a TERT promoter, said method comprising:
(a) contacting said agent with a normal cell under assay conditions that provide for a detectable phenotype upon modulation of TERT promoter transcription control activity; and
(b) evaluating said cell for said detectable phenotype to determine whether said agent modulates transcription control activity of said TERT promoter nucleic acid.

2. The method according to claim 1, wherein said cell is a normal cell and said method is a method of determining whether said agent enhances expression controlled by a TERT promoter nucleic acid.

3. The method according to claim 2, wherein said detectable phenotype is cell death.

4. The method according to claim 3, wherein said cell undergoes cell death if said agent enhances expression controlled by a TERT promoter nucleic acid.

5. The method according to claim 3, wherein said cell undergoes cell death if said agent does not enhance expression controlled by a TERT promoter nucleic acid.

6. The method according to claim 1, wherein said TERT promoter nucleic acid is a human TERT promoter nucleic acid.

7. The method according to claim 6, wherein said cell is a human cell.

8. The method according to claim 1, wherein said method comprises determining the modulatory activity of at least two different agents.

9. The method according to claim 8, wherein said method is a high-throughput method.

10. The method according to claim 1, wherein said agent is a small molecule.

11. A method of determining whether a small molecule agent can derepress transcription repression activity of a TERT promoter, said method comprising:
(a) contacting said agent with a normal cell under assay conditions that provide for cell death in the presence of telomerase reverse transcriptase (TERT); and
(b) evaluating said cell for cell death to determine whether said agent derepresses transcription repression activity of said TERT promoter nucleic acid.

12. The method according to claim 11, wherein said cell is a human cell.

13. The method according to claim 12, wherein said TERT promoter nucleic acid is a human TERT promoter nucleic acid.

14. The method according to claim 11, wherein said method comprises determining the activity of at least two different agents.

15. The method according to claim 14, wherein said method is a high-throughput method.

16. The method according to claim 11, wherein said agent is a small molecule.

17. A method of determining whether a small molecule agent can derepress transcription repression activity of a TERT promoter, said method comprising:
(a) contacting said agent with a normal cell under assay conditions that provide for cell death in the absence of telomerase reverse transcriptase (TERT); and
(b) evaluating said cell for cell death to determine whether said agent derepresses transcription repression activity of said TERT promoter nucleic acid.

18. The method according to claim 17, wherein said cell is a human cell.

19. The method according to claim 18, wherein said TERT promoter nucleic acid is a human TERT promoter nucleic acid.

20. The method according to claim 17, wherein said method comprises determining the activity of at least two different agents.

21.-25. (canceled)
